(19) United States
(12) Patent Application Publication
(10) Pub. No.: US 2025/0059138 A1
(43) Pub. Date: Feb. 20, 2025

(54) CHEMICAL LINKERS

(71) Applicant: Sigma-Aldrich Co. LLC, St. Louis, MO (US)

(72) Inventors: Ravindra Vikram Singh, Bangalore (IN); Saikat Sinha, Bangalore (IN); Rhushikesh Chandrabhan Deokar, Bengaluru (IN); Venkata Ganesh Akula, Bengaluru (IN); Davinder Prasad, Bangalore (IN); Pratik Swarup Gucchait, Bangalore (IN); Sateesh Madhi, Germantown, WI (US); Kaelyn Wilke, Wauwatosa, WI (US)

(21) Appl. No.: 18/722,011

(22) PCT Filed: Dec. 15, 2022
(86) PCT No.: PCT/US2022/081681

§ 371 Date: Jun. 20, 2024

Related U.S. Application Data
(60) Provisional application No. 63/265,717, filed on Dec. 20, 2021.

(51) Int. Cl. C07D 213/64; C07D 213/74; C07D 239/42; C07D 295/205; C07D 295/215; C07D 401/04

(52) U.S. Cl. CPC C07D 213/64; C07D 213/74; C07D 239/42; C07D 295/205; C07D 295/215; C07D 401/04

(57) ABSTRACT

Heterobifunctional chemical linkers useful in protein degraders and other medicinal chemistry applications are provided. The heterobifunctional linkers include alky ne-piperidine and alkane-piperidine linkers: alkane-piperazine linkers; alkanamino PEGylated linkers; dipiperazine linkers; piperazine-pyridine/pyrimidine-alkyne linkers; pyridine-PEG-piperazine linkers and pyrimidine-piperazine-PEG linkers. Also provided are combinations of the provided linkers with a ligand that targets a degradation pathway, such as an E3 ubiquitin ligase enzyme.

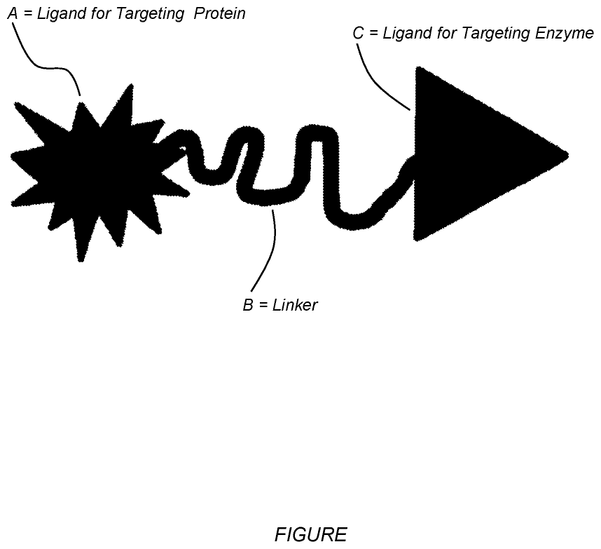

### CROSS-REFERENCE TO RELATED APPLICATIONS

The present application claims the benefit of priority of U.S. Provisional Application No. 63/265,717, filed Dec. 20, 2021, the entire contents of which is incorporated herein by reference.

### BACKGROUND

Protein degraders are heterobifunctional molecules including a ligand targeting a protein of interest (A) at one end; a ligand targeting a ligase or other enzyme (C) useful in degrading the protein of interest, the other end; and a linker (B), connecting the protein targeting ligand with the enzyme targeting ligand. Much of the research on such protein degraders has focused on the ligand targeting the ligase, specifically those targeting an E3 ubiquitin ligase enzyme. Another focus of research has been combinations of protein targeting ligands and ligase targeting ligands. The choice of linker, however, has been less studied.

While less studied, the choice of linker is important to these

heterobifunctional protein degraders. The linker length and pole affect, for example, cell permeability and solubility. A need exists for new linkers that can provide tailored properties to the heterobifunctional protein degraders. Because of the myriad of potential applications there is a need for new linkers that can provide specific properties, such as lipophilicity or hydrophilicity, high solubility, and rigidity or flexibility.

### SUMMARY

Provided are chemical linkers useful in protein degraders and other applications. In a first embodiment, the chemical linkers are those of Formula I:

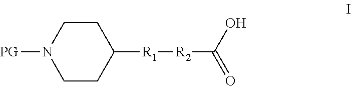

wherein R₁ is optional, and if present, is selected from piperidine and piperazine, R₂ is selected from C₁-C₈ alkane and C₂-C₈ alkyne, and PG is an amine protecting group.

A second embodiment provided are linkers Formula II:

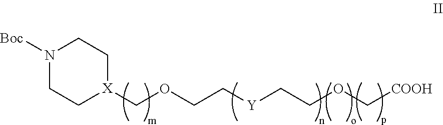

wherein X is C or N; m is 1 to 6; Y is —O— or —CH₂—, n is from 0 to 4, o is 0 or 1, and p is from 0 to 9.

A third embodiment provided are linkers of Formula III:

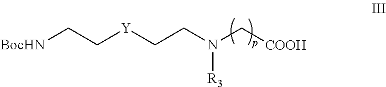

wherein Y is —O— or —CH₂—; R₃ is C₁-C₆ alkyl, C₃-C₆ cycloalkyl, or cycloalkyl-substituted alkyl; and p is 1 or 2.

A fourth embodiment provided are linkers of Formula IV:

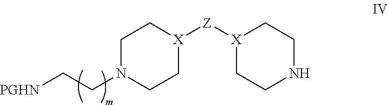

wherein PG is an amine protecting group; m is 1-4; each X is independently C or N; and Z is selected from —C(O)—, —C(S)—, —CH₂—, or —CF₂—.

A fifth embodiment provided are linkers of Formula V:

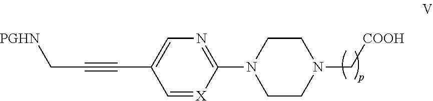

wherein PG is an amine protecting group, X is N or C, and p is 1-3.

A sixth embodiment provided are linkers of Formula VI:

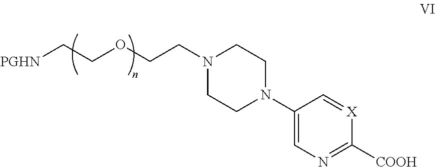

wherein PG is an amine protecting group; each X is independently C or N; and n is 0-2.

A seventh embodiment provided are linkers of Formula VII:

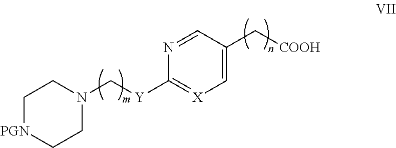

wherein PG is an amine protecting group; X is C or N; Y is N, O or C; m is 1-3; and n is 1-3.

### BRIEF DESCRIPTION OF THE DRAWING

The FIGURE is an illustration depicting a protein degrader including (A) a ligand targeting a protein of interest, (B) a connecting linker, and (C) a ligand targeting an enzyme.

### DETAILED DESCRIPTION

Protein degraders are combination of three components: A, a ligand for the target protein, B, a linker molecule and C a handle, or ligand for an enzyme, such as E3 ubiquitin ligase enzyme. New heterobifunctional chemical linkers of Formulas I-VII are provided. The disclosed linkers are particularly useful in the fields of targeted protein degradation and targeted drug delivery. One exemplary use is in protein degraders, i.e., proteolysis targeting chimeras (PROTACs). Such protein degraders include a ligand specific to a target protein (A), a handle for an E3 ubiquitin ligase enzyme (C), and a linker (B) linking the target protein-specific ligand to the E3 ubiquitin ligase enzyme, as illustrated graphically in the FIGURE. The linkers disclosed herein are designed to have hydrophobic or hydrophilic functionality, high solubility, and varying degrees of rigidity and/or flexibility, making these linkers suitable for a wide range of applications. Also provided are protein degrader building blocks including the chemical linker attached to a handle for an E3 ubiquitin ligase enzyme. Such protein degrader building blocks are ready to conjugate to a target protein ligand. The linkers provided herein are also useful in other applications, including targeted drug delivery and discovery.

In a first embodiment, the chemical linkers are those of Formula I:

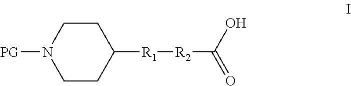

wherein R₁ is optional, and if present, is selected from piperidine and piperazine, R₂ is selected from C₁-C₈ alkane, i.e., —CH₂— to —C₈H₁₆— and C₂-C₈ alkyne, e.g., —C≡C— or —C₂— to —C₈H₁₂—, and PG is an amine protecting group. In some embodiments, the R₂ is selected from ethane (—C₂H₄—) and ethyne (—C≡C—). In some embodiments, protecting group PG is tert-butoxycarbonyl (tert-butyloxycarbonyl, Boc).

In some preferred embodiments, the linker of formula I is one of

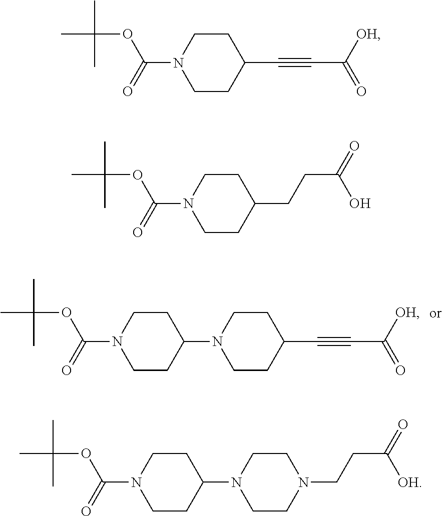 [SMILES: CC(C)(C)OC(=O)N1CCC(C#CC(=O)O)CC1 ; CC(C)(C)OC(=O)N1CCC(CCC(=O)O)CC1 ; CC(C)(C)OC(=O)N1CCC(N2CCC(C#CC(=O)O)CC2)CC1 ; CC(C)(C)OC(=O)N1CCC(N2CCN(CCC(=O)O)CC2)CC1]

In a second embodiment, provided are linkers of Formula II:

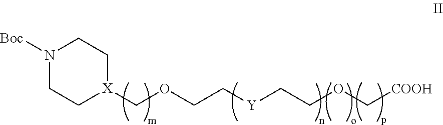

wherein X is selected from C and N; m is 1 to 6; Y is selected from —O— and —CH₂—, n is from 0 to 4, o is 0 or 1, and p is 0 to 9.

In some embodiments, the linker of Formula II is, is that of Formula IIa:

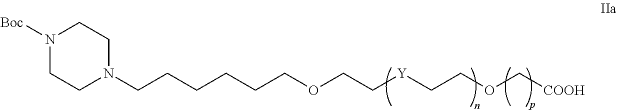

wherein Y is selected from —CH₂— and —O—; n is 0 to 5, and p is 1 to 5. In some embodiments of Formula IIa, n is 0, 1, or 4. In some embodiments of Formula IIa p is 1 or 5.

In other embodiments, the linker of Formula II is that of Formula IIb:

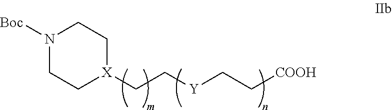

wherein X is selected from C and N, Y is selected from —CH₂— and —O—; m is 0 or 1, and n is 0, 1, 2, or 3.

In still other embodiments, the linker of Formula II is that of Formula IIc:

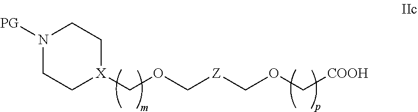

wherein X is selected from C and N; Z is selected from —CH₂—, —CF₂—, —C(O)—, and —C(S)—; m is 2 to 5; p is 1 or 2; and PG is an amine protecting group. In some embodiments, m is 2, 3 or 5. In some embodiments, a preferred amine protecting group, PG, is Boc.

Also provided are linkers of Formula III:

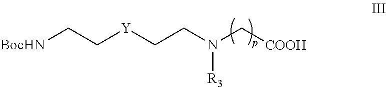

wherein Y is selected from —O— and —CH₂—; R₃ is selected from C₁-C₆ alkyl, C₃-C₆ cycloalkyl, and cycloalkyl-substituted alkyl, and p is 1 or 2. In various embodiments, R₃ is selected from methyl, ethyl, isopropyl, tert-butyl, n-butyl, cyclopropyl, cyclobutyl, cyclopentyl, cyclohexyl, and methylenecyclobutyl

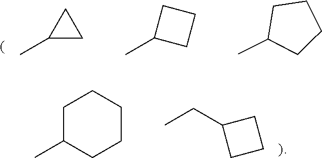

Also provided are linkers of Formula IV:

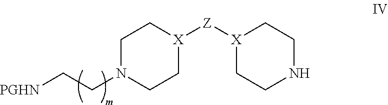

wherein PG is an amine protecting group; m is 1-4; each X is independently selected from C and N; and Z is selected from —C(O)—, —C(S)—, —CH₂—, and —CF₂—. In some embodiments, the preferred amine protecting group, PG, is Boc. In some embodiments, both X are C. In other embodiments, both X are N. In still other embodiments, one X is C and one X is N.

Also provided are linkers of Formula V:

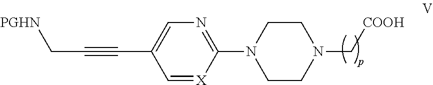

wherein PG is an amine protecting group, X is N or C, and p is 1-3. In some embodiments, the preferred amine protecting group, PG, is Boc.

Also provided are linkers of Formula VI:

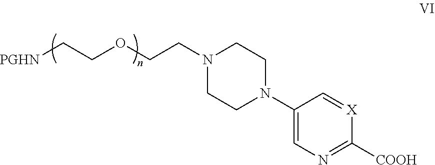

wherein PG is an amine protecting group; X is C or N; and n is 0-2. In some embodiments, the preferred amine protecting group, PG, is Boc.

Also provided are linkers of Formula VII:

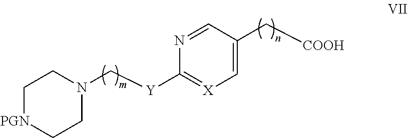

wherein PG is an amine protecting group; X is C or N; Y is selected from N, O and C; m is 1-3; and n is 1-3. In some embodiments, the preferred amine protecting group, PG, is Boc.

The linkers of Formulas I-VII are useful in myriad applications, such as in protein degraders, as exemplified in the FIGURE. The linker length and pole composition of protein degraders affect characteristics such as cell permeability, and solubility. The linkers provided herein offer a mixture of hydrophobic and hydrophilic functionality to balance the hydrophobicity-hydrophilicity of the resulting hybrid compounds. These linkers, when combined with various ligand for an E3 ubiquitin ligase enzyme, can be used to generate a large variety of degrader building blocks (B+C). Using these degrader building blocks, and combining this with ligand for the target protein (A), one can generate large protein degrader libraries of their interest, enabling screening of a large number of compounds for screening and potentially identification of new drugs.

Further provided are compounds having a linker of Formula I-VII and a ligand that triggers a degradation pathway. In some embodiments, the ligand that triggers a degradation pathway is an E3 ubiquitin ligase enzyme. In some embodiments, the ligand for an E3 ubiquitin ligase enzyme is a derivative of an immunomodulatory drug (IMiD). In some embodiments, the derivative of an IMiD is selected from pomalidomide derivatives, thalidomide derivatives, lenalidomide derivatives, and VH032 derivatives.

The linkers provided herein are also useful to researchers working in other therapeutic areas, and in medicinal chemistry projects. The provided linkers are useful in the design and function of bioactive molecules and the design of targeted drug delivery & discovery.

### EXAMPLES

### Example 1. Preparation of a Representative Alkyne-Piperidine Linker

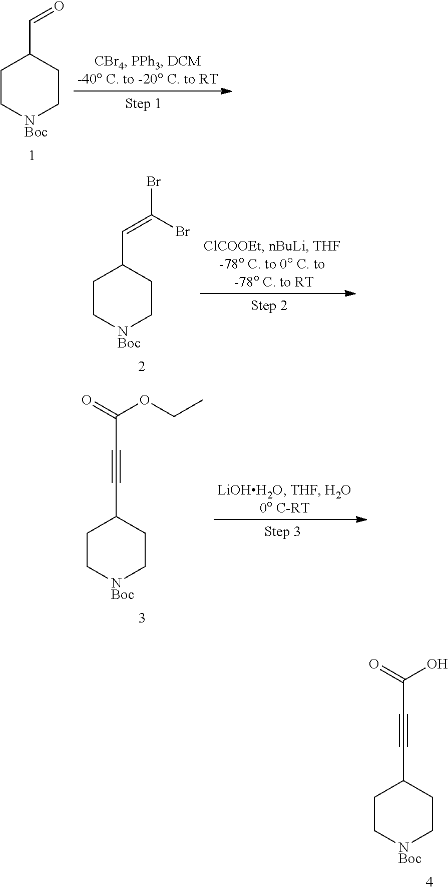 [SMILES: CC(C)(C)OC(=O)N1CCC(C=O)CC1 ; CC(C)(C)OC(=O)N1CCC(C=C(Br)Br)CC1 ; CCOC(=O)C#CC1CCN(C(=O)OC(C)(C)C)CC1 ; CC(C)(C)OC(=O)N1CCC(C#CC(=O)O)CC1]

### Step 1: Synthesis of tert-butyl 4-(2,2-dibromoethenyl)piperidine-1-carboxylate

Triphenylphosphine (123.0 g, 468.8 mmol, 4.0 eq) and tetrabromomethane (77.76 g, 234.42 mmol, 2.0 eq) were charged in 3 L four neck round bottom (RB) flask, equipped with a magnetic bar, thermometer, 500 mL pressure equalizing addition funnel topped with a N₂ inlet adapter. Then the reaction mass was cooled-30° C. (±5° C.) by dry ice-isopropyl alcohol (IPA) mixture with stirring under nitrogen atmosphere. Dichloromethane (DCM) (500 ml) was added slowly over a period of 45 min via addition funnel at −20° C. under nitrogen atmosphere while stirring vigorously. Reaction mass was stirred for 15 min at −20° C., followed by slow addition of 1-bocpiperidine-4-carboxaldehyde 1 (25.0 g, 117.21 mmol, 1.0 eq) dissolved in DCM (125 ml) over a period of 2 h at the temperature −20° C. (±5° C.) with a vigorous stirring condition. Then the reaction mass was stirred for 18 h at room temperature under nitrogen atmosphere. After completion of reaction (checked by TLC, in 10% ethyl acetate/hexane; R_{f}: 0.5), the reaction mass was filtered through sintered funnel, residue was washed with DCM (100 ml). Filtrate was concentrated to dryness. Concentrated fraction was diluted with hexane (1 L) to make a suspension. Suspension was filtered through sintered funnel, washed the residue with hexane (200 ml). Filtrate was concentrated at 40° C. Suspension preparation, filtration, followed by filtrate concentration was repeated two times to remove the unwanted polar impurities (triphenylphosphine oxide). Finally, the compound was dried under vacuum to get the off-white solid dibromoalkene 2 (24 g, 79% yield). ¹H NMR (CDCl₃, 400 MHz): δ 6.23 (d, J=8.8 Hz, 2H), 4.05 (brs, 2H), 2.79 (t, J=11.6 Hz, 2H), 2.45-2.39 (m, 1H), 1.70-1.66 (m, 2H), 1.46 (s, 9H), 1.35-1.28 (m, 2H).

### Step 2: Synthesis of tert-butyl 4-(3-ethoxy-3-oxoprop-1-yn-1-yl)piperidine-1-carboxylate

tert-butyl 4-(2,2-dibromoethenyl) piperidine-1-carboxylate 2 (15.0 g, 40.64 mmol, 1.0 eq), and tetrahydrofuran (THF) (300 ml) were taken in an 1 L four-necked RB flask with a magnetic bar, cooling bath, thermometer, two 500 ml pressure equalizing addition funnels topped with a N₂ inlet adapter. Then the reaction mass was cooled to −78° C. by dry ice acetone bath under nitrogen atmosphere followed by dropwise addition of 1.4 M n-butyl lithium (84 ml, 117.86 mmol, 2.9 eq) through liquid addition funnel over a period of 1 h at −78° C. under nitrogen atmosphere. Then the reaction mass was stirred for 1 h at −78° C. and next 1 h at 0° C. under nitrogen atmosphere. The reaction mass was cooled to −78° C. and ethyl chloroformate (14.4 ml, 150.37 mmol, 3.7 eq) was added dropwise to the reaction mass over a period of 30 min under nitrogen atmosphere. The reaction mass was warmed to 25° C. over a period of 2 h and completion of reaction was checked by thin layer chromatography (TLC) (30% ethyl acetate in Hexane, R_{f}=0.6, visualization after charging with phosphomolybdic acid (PMA) TLC stain). The reaction mixture was quenched carefully with the dropwise addition of ice-cold water (15 ml) at −78° C. (±2° C.) and diluted carefully with the dropwise addition of saturated NaCl solution (200 ml) at −78° C. (±2° C.), extracted ethyl acetate (250 mL×3). Combined organic layers were dried layers over anhydrous sodium sulfate (50 g), filtered and concentrated at 42° C. under reduced pressure to dryness to afford crude product. The crude material was purified by silica gel (60-120 mesh) column chromatography with 4% ethyl acetate-hexane. Pure fractions were combined and concentrated at 42° C. under reduced pressure to dryness to get the ester 3 (6.5 g, 57% yield) as light brown oily liquid. ¹H NMR (CDCl₃, 400 MHz): δ 4.20-4.15 (m, 2H), 3.69-3.65 (m, 2H), 3.17-3.11 (m, 2H), 2.69-2.65 (m, 1H), 1.81-1.75 (m, 2H), 1.64-1.62 (m, 2H), 1.40 (s,9H), 1.30-1.25 (m, 3H).

### Step 3: Synthesis of 1-piperidinecarboxylic acid, 4-(2-carboxyethynyl)-, 1-(1,1-dimethylethyl) ester

A 500 mL four necked RB flask was equipped with a magnetic bar, thermometer and a N₂ inlet adapter. Tert-butyl 4-(3-ethoxy-3-oxoprop-1-yn-1-yl) piperidine-1-carboxylate 3 (6.50 g, 23.10 mmol, 1 eq), THF (65 mL) and water (26 mL) were added into it. Then the reaction mass was cooled to 0°-5° C. by ice bath. Lithium hydroxide monohydrate (1.94 g, 46.23 mmol, 2 eq) in one lot. Then the reaction mass was stirred for 15 h at 25° C. After completion of reaction (checked by TLC, in 10% methanol/DCM; R_{f}: 0.1), the reaction mass was concentrated to remove the volatile solvent and the residue was washed with ethyl acetate (3×100 ml). The ethyl acetate fraction was discarded, and the aq. fraction was acidified with saturated aq. solution of citric acid up to pH ˜3. Then the aq. fraction was extracted with ethyl acetate (3×250 ml). Combined organics were washed with water (3×100 ml) and brine (2×100 ml). Organic layer was dried over anhydrous sodium sulfate (50 g), filtered and concentrated at 42° C. under reduced pressure to dryness to afford crude product. The crude material was washed by n-hexane (200 ml), filtered and dried by vacuum to get the acid 4 (4.50 g, 77% yield) as off white solid. ¹H NMR (CDCl₃, 400 MHz): δ 8.50 (bs, 1H), 3.71-3.68 (m, 2H), 3.23-3.16 (m, 2H), 2.74-2.72 (m, 1H), 1.84-1.81 (m, 2H), 1.68-1.63 (m, 2H), 1.44 (s,9H).

### Example 2. Synthesis of a Representative Alkane-Piperazine Linker

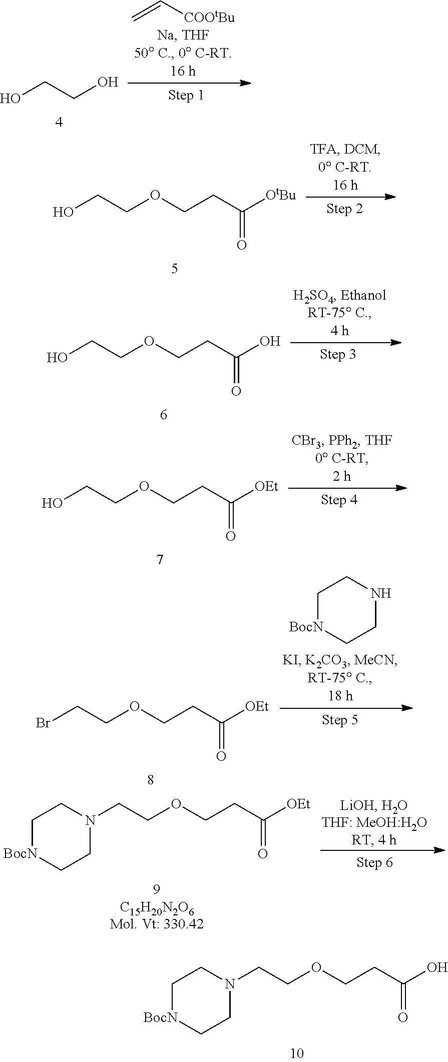

### Step 1: Synthesis of tert-butyl 3-(2-hydroxyethoxy) propanoate

To the reaction flask was added ethylene glycol (125.0 g, 2.01 mol, 1.0 eq) in 1000 ml of dry THF with stirring. Then portion-wise sodium metal (1.38 g, 60.0 mmol, 0.03 eq.) was added into the reaction flask and heated the reaction mixture at 50° C. for 4 h to obtain a clear solution. The reaction mixture was cooled to 0° C. and dropwise added a solution of tert-Butyl acrylate (206.1 g, 1.61 mol, 0.8 eq.) in 300 ml of dry THF over a period of 1 h. Then the reaction mixture was allowed to stir at room temperature for 15-16 hours. The progress of the reaction was monitored by TLC (30% ethyl acetate in hexane; Rf: 0.25). The reaction mixture was diluted with 1.5 L of ethyl acetate and the organic layer was washed with ice-cold water, sat. NaHCO₃ and brine. The organic layer was concentrated on rotary evaporator to yield crude product as pale green gel which was then purified through column chromatography using 100-200 mesh silica gel and eluted the product with 10-15% of ethyl acetate in hexane. Combined fractions were evaporated to yield pure tert-butyl 3-(2-hydroxyethoxy) propanoate 5 (68.0 g, 18% yield) as light greenish liquid. ¹H NMR (CDCl₃, 400 MHz): δ 3.75 (t, 4H), 3.62 (t, 2H), 2.52 (t, 2H), 1.43 (s, 9H).

### Step 2: Synthesis of 3-(2-hydroxyethoxy) propanoic acid

In a 1 L RB flask tert-butyl 3-(2-hydroxyethoxy) propanoate 5 (68.0 g, 357.4 mmol, 1.0 eq) was charged in 400 ml DCM. The solution was cooled to 0° C. and then a solution of TFA (108 ml) in 200 ml of DCM was added through addition funnel over a period of 30 minutes. Then the reaction mixture was allowed to stir at room temperature for 15-16 hours. The progress of the reaction was monitored by TLC (5% methanol in dichloromethane; Rf: 0.1). After completion, the reaction mixture was evaporated to dryness using rotary evaporator to yield thick gel. The crude material was dissolved into 20 mL of 10% methanol in DCM and triturated with n-hexane (250 ml×2). Hexane layer then decanted and residue was evaporated to yield light brown gel as tert-butyl 3-(2-hydroxyethoxy) propanoic acid 6 (44.0 g, 92% yield). ¹H NMR (CDCl₃, 400 MHz): δ 3.75 (t, 4H), 3.62 (t, 2H), 2.32 (t, 2H), 11.2 (brs, 1H)

### Step 3: Synthesis of ethyl 3-(2-hydroxyethoxy) propanoate

1000 ml four necked RB flask was equipped with refluxing condenser, N₂ inlet adapter and 3-(2-hydroxyethoxy) propanoic acid 6 (40.0 g, 198.2 mmol, 1.0 eq) was dissolved in 600 ml of ethanol into the reaction flask with stirring. Conc. H₂SO₄ (12.5 ml) was then added to the reaction mixture dropwise and the reaction mixture was refluxed at 75° C. for 4 hours. After completion (checked by TLC in 5% methanol in dichloromethane; Rf: 0.6), the reaction mixture was cooled to 30° C. (±2) and diluted with 1.5 L of DCM, washed the organic layer with cold water (1000 ml×2) and sat. NaHCO₃ solution (1000 ml). Evaporation of the organic layer to dryness using rotary evaporator afforded pale yellow liquid. The crude material was purified using silica gel (100-200 mesh) column chromatography using 2% methanol in DCM as eluent. Combined fractions containing pure product were evaporated at 40° C. under reduced pressure to dryness to yield ethyl 3-(2-hydroxyethoxy) propanoate 7 as pale yellow liquid (25 g, 52% yield). The crude product was taken to next step without any purification. ¹H NMR (CDCl₃, 400 MHz): δ 4.02 (q, 2H), 3.75 (t, 4H), 3.62 (t, 2H), 2.52 (t, 2H), 1.23 (t, 3H), LCMS (ESI, m/z): 163.0 (M+H)⁺.

### Step 4: Synthesis of ethyl 3-(2-bromoethoxy) propanoate

2 L four necked RB flask was equipped with a magnetic bar, 500 ml addition funnel and a N₂ inlet adapter and ethyl 3-(2-hydroxyethoxy) propanoate 7 (25.0 g, 154.1 mmol, 1.0 eq) in 600 ml of dry THF was added into the reaction flask with stirring. Triphenylphosphine (60.6 g, 231.2 mmol, 1.5 eq.) was added to the reaction mixture in one portion and allowed to stir the reaction mixture at room temperature for 10-15 minutes to obtain a clear solution. The reaction mixture was cooled to 0° C. and a solution of CBr₄ (76.7 g, 231.2 mmol, 1.5 eq) in 400 ml of dry THF was added dropwise to the reaction mixture over a period of 30 minutes. Then allowed to stir the reaction mixture at room temperature for 2-3 hours. The progress of the reaction was monitored by TLC (30% ethyl acetate in hexane; Rf: 0.7). The white precipitate was filtered off through filter paper and washed with THF (100 ml) and filtrate was evaporated to yield sticky precipitate. Crude material was then purified using silica gel (60-120 mesh) column chromatography in 30% ethyl acetate in hexane as eluent. Combined pure fractions containing pure product were evaporated at 40° C. under reduced pressure to dryness to yield ethyl 3-(2-bromoethoxy) propanoate 8 (21 g, 61% yield) as gummy liquid. ¹H NMR (CDCl₃, 400 MHz): δ 4.02 (q, 2H), 3.75 (t, 4H), 3.43 (t, 2H), 2.62 (t, 2H), 1.43 (s, 9H), 1.23 (t, 3H)

### Step 5: Synthesis of tert-butyl 4-(2-(3-ethoxy-3-oxopropoxy)ethyl)piperazine-1-carboxylate

1000 mL four necked RB flask was equipped with a magnetic bar, refluxing condenser and a N₂ inlet adapter and ethyl 3-(2-bromoethoxy) propanoate 8 (14.6 g, 64.9 mmol, 1.10 eq) was charged in 600 ml of acetonitrile into the reaction flask with stirring. To this reaction mixture then Boc-piperazine (11.0 g, 58.9 mmol, 1.0 eq.) was added in one portion followed by the addition of potassium carbonate (20.4 g, 147.3 mmol, 2.5 eq). Then KI (2.4 g, 14.7 mmol 0.25 eq) was added and the reaction mixture was refluxed at 82±2° C. for 18 h. The Progress of the reaction was monitored by TLC (5% methanol in Dichloromethane; Rf: 0.3) and the reaction mixture was cooled to RT, the white precipitate was filtered off through cellite bed and washed with acetonitrile (100 ml). Filtrate was evaporated to dryness and purified the crude compound through column chromatography (60-120 mesh silica gel) using 2-3% methanol in dichloromethane as eluent. Combined organic fractions containing pure product were evaporated at 40° C. under reduced pressure to dryness to yield tert-butyl 4-(2-(2-(ethoxycarbonyl)ethoxy)ethyl)piperazine-1-carboxylate 9 (15 g, 77% yield) as pale yellow solid. ¹H NMR (CDCl₃, 400 MHZ): δ 4.02 (q, 2H), 3.65 (t, 4H), 3.43 (t, 4H), 2.72 (t, 2H), 2.52 (t, 4H), 2.45 (t, 2H), 1.43 (s, 9H), 1.23 (t, 3H).

### Step 6: Synthesis of 3-(2-(4-(tert-butoxycarbonyl)piperazin-1-yl)ethoxy)propanoic acid

Tert-butyl 4-(2-(2-(ethoxycarbonyl)ethoxy)ethyl)piperazine-1-carboxylate 9 (15.0 g, 45.4 mmol, 1.0 eq.) was dissolved in a mixture of THF-water (1:1, 400 ml). Lithium hydroxide monohydrate (5.7 g, 136.2 mmol, 3.0 eq.) was added into the reaction mixture and allowed the reaction mixture to stir at RT for 4-5 hours. Reaction completion was checked by TLC monitoring (20% methanol in dichloromethane; Rf; 0.015). The reaction mixture was diluted with 500 ml of ethyl acetate and separated organic layer was discarded. The aqueous layer was acidified with 1N citric acid solution to PH˜6 (±0.2) using pH meter and lyophilize the aqueous layer for 18 h to yield off white solid. Crude material was then subjected to silica gel (60-120 mesh size) column chromatography using 8-10% methanol in dichloromethane as eluent. Combined fractions containing pure product were evaporated under reduced pressure to dryness to yield 10 4-(2-(2-(ethoxycarbonyl)ethoxy)ethyl)piperazine-1-carboxylic acid (6.6 g, 48% yield) as white solid flakes. ¹H NMR (D₆-DMSO, 400 MHz): δ 10.5-13.10 (brs, 1H), 3.65 (t, 4H), 3.43 (t, 4H), 2.72 (t, 2H), 2.52 (t, 4H), 2.45 (t, 2H), 1.43 (s, 9H).

### Example 3. Preparation of a Representative Alkanamino PEGylated Linker

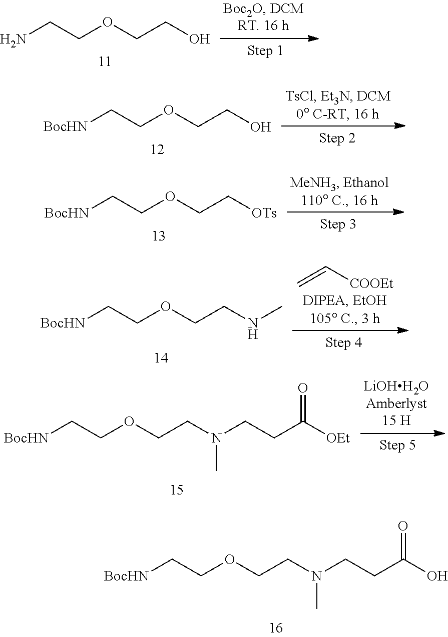

### Step 1: Synthesis of tert-butyl N-[2-(2-hydroxyethoxy)ethyl]carbamate

A solution of di-tert-butyl dicarbonate (32.0 g, 148.0 mmol, 1.1 eq) in methylene chloride (70 ml) was added dropwise to a solution of 3-amino-1-propanol 11 (10.1 g, 134.0 mmol, 1.0 eq) in methylene chloride (100 ml). The reaction mixture was stirred overnight and was washed with saturated aqueous sodium bicarbonate solution, water and then brine. The organic layer was dried (Na₂SO₄) and an organic layer were evaporated on rotary evaporator to give 3-(N-tert-butoxycarbonylamino)-1-propanol 12 (23 g, 98% yield) as a colorless oil. ¹H NMR (CDCl₃, 400 MHz): δ 4.78 (brs, 1H), 3.65 (m, 2H), 3.30 (m, 2H), 2.90 (brs, 1H), 1.68 (m, 2H), 1.48 (s, 9H).

### Step 2: Synthesis of 2-[2-(tert-butoxycarbonylamino)ethoxy]ethyl 4-methylbenzenesulfonate

A solution of tert-butyl N-[2-(2-hydroxyethoxy)ethyl]carbamate 12 (20.0 g, 114.0 mmol, 1.0 eq) in methylene chloride was added triethylamine (31.8 ml, 228.0 mmol, 2.0 eq) at 0° C. with ice cooled bath. P-toluenesulfonyl chloride (32.6 g, 171.0 mmol, 1.5 eq) was added portion wise over a period of 15 min time. Then allowed the reaction mixture was stirred overnight and was then quenched with the cold water (100 ml). The organic layer was dried (Na₂SO₄) and the volatiles were removed by evaporation to give gummy residue which was purified by column chromatography by using silica 60-120 mesh, compound was eluted in 10% ethyl acetate/hexane to afford 2-[2-(tert-butoxycarbonylamino)ethoxy]ethyl 4-methylbenzenesulfonate 13 (12 g, 32% yield) as a pale yellow liquid. ¹H NMR (400 MHz, CDCl₃): δ ¹ H NMR (400 MHz, CDCl₃): δ 3.58 (t, J=5.09 Hz, 2H), 3.51 (t, J=5.24 Hz, 2H), 3.34-3.27 (m, 2H), 2.78 (t, J=4.89 Hz, 2H), 1.44 (s, 9H).

### Step 3: Synthesis of 1,1-dimethylethyl N-[2-[2-(methylamino)ethoxy]ethyl]carbamate

A solution of 2-[2-(tert-butoxycarbonylamino)ethoxy]ethyl 4-methylbenzenesulfonate 13 (8.0 g, 24.3 mmol, 1.0 eq) in EtOH (40 ml) in seal tube was added methyl amine (10 mL, 33 wt. % in absolute ethanol) at room temperature, the seal tube was sealed and placed in oil bath. The reaction mixture heated to 110° C. and the reaction mixture was stirred overnight. Reaction mixture was then evaporated under reduced pressure to give gummy residue, which was purified by silica gel column chromatography, eluted in 70% Ethyl acetate/Hexane to afford 1,1-Dimethylethyl N-[2-[2-(methylamino)ethoxy]ethyl]carbamate 14 (4.3 g, 98% yield) as colourless liquid. ¹ H NMR (400 MHz, CDCl₃): δ 4.10-4.08 (m, 2H), 3.57-3.54 (m, 2H), 3.3 3.37 (m, 2H), 3.17-3.16 (m, 2H), 2.38 (s, 3H), 1.37 (s, 9H).

### Step 4: Synthesis of ethyl 3-{[2-(2-{[(tert-butoxy)carbonyl]amino}ethoxy)ethyl](methyl)amino}propanoate

A solution of 1,1-Dimethylethyl N-[2-[2-(methylamino)ethoxy]ethyl]carbamate 14 (1.0 g, 5.31 mmol, 1.0) in THF (10 ml) was added N,N-diisopropylethylamine (2.8 ml, 15.9 mmol, 3.0) and ethyl acrylate (1.0 g, 10.6 mmol, 2.0) at 0° C. The reaction mass was stirred at 0° C. for 10 min then slowly heated to 105° C. and heated at the same temperature for 3 h. The reaction was quenched with water and extracted with DCM for 3 times. The organic layer was dried (Na₂SO₄) and the volatiles were removed by evaporation to give crude 4-benzyl 7-ethyl 8-oxo-4-azaspiro [2.5] octane-4,7-dicarboxylate which was purified by column chromatography by using silica 100-200 mesh, compound was eluted in 5% MeOH/DCM to afford pure ethyl 3-{[2-(2-{[(tert-butoxy)carbonyl]amino}ethoxy)ethyl](methyl)amino}propanoate 15 (0.48 g, 31% yield) as colorless liquid. ¹ H NMR (400 MHz, CDCl₃): δ 4.07 (q, J=7.17 Hz, 2H), 3.52-3.41 (m, 4H), 3.26-3.19 (m, 2H), 2.71 (t, J=7.46 Hz, 2H), 2.53 (t, J=5.45 Hz, 2H), 2.42 (t, J=7.26 Hz, 2H), 2.22 (s, 3H), 1.37 (s, 9H), 1.18 (t, J=7.34, 3H).

### Step 5: Synthesis of 3-{[2-(2-{[(tert-butoxy)carbonyl]amino}ethoxy)ethyl](methyl)amino}propanoic acid

A solution of ethyl 3-{[2-(2-{[(tert-butoxy)carbonyl]amino}ethoxy)ethyl](methyl)amino}propanoate 15 (10 g, 34.7 mmol, 1.0 eq) in MeOH-THF-H₂O (1:1:1, 30 ml) was added LiOH·H₂O (1.7 g, 69.4 mmol, 2.0 eq) at room temperature. The reaction mixture was stirred at room temperature for 18 h. Solvent was evaporated under reduced pressure and extracted with DCM for 3 times. The organic layer was dried (Na₂SO₄) and the organic layer was evaporated on rotary evaporator to give crude product as a lithium salt of carboxylic acid. The salt was then redissolved in distilled water and further treated with Amberlyst-15 hydrogen ion resin. The pH of the aqueous phases was adjusted to PH ˜5 using pH meter. The resin was removed by filtered and water layer was lyophilized which produced 3-{[2-(2-{[(tert-butoxy)carbonyl]amino}ethoxy)ethyl](methyl)amino}propanoic acid 16 (3.8 g, 42% yield) as white gummy hygroscopic solid. ¹H NMR (400 MHz, CDCl₃): δ 3.57-3.51 (m, 4H), 3.32-3.30 (m, 2H), 2.69-2.65 (m, 2H), 2.60-2.56 (m, 2H), 2.38-2.34 (m, 2H), 2.16 (s, 3H), 1.37 (s, 9H).

### Example 4. Preparation of a Representative Dipiperazine Linker

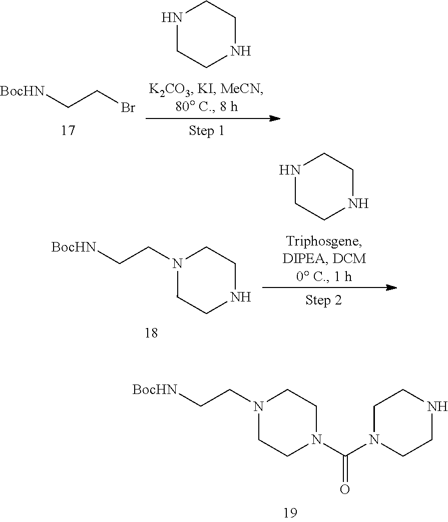 [SMILES: C1CNCCN1 ; CC(C)(C)OC(=O)NCCBr ; C1CNCCN1 ; CC(C)(C)OC(=O)NCCN1CCNCC1 ; CC(C)(C)OC(=O)NCCN1CCN(C(=O)N2CCNCC2)CC1]

### Step 1: Synthesis of tert-butyl (2-(piperazin-1-yl)ethyl)carbamate

A 2000 mL three necked RB flask was equipped with a magnetic bar, refluxing condenser and a N₂ inlet adapter. Tert-butyl (2-bromoethyl) carbamate 17 (24.4 g, 108.9 mmol, 1.0 eq) was added in 1200 mL of acetonitrile. Piperazine (37.5 g, 435.7 mmol, 4.0 eq) was added followed by K₂CO₃ (45.2 g, 326.7 mmol, 3.0 eq) and KI (18.1 g, 108.9 mmol, 1.0 eq) into the reaction flask with stirring under N₂ atmosphere. The reaction mixture was stirred at room temperature for 20 minutes. The reaction mixture was stirred at 80° C. for 10 hours. The progress of the reaction was monitored by TLC (in 10% MeOH: DCM, R_{f}: 0.2). After completion, the reaction mixture was cooled to room temperature and acetonitrile was evaporated and the residue was diluted with DCM (1 L). The organic layer was washed with water (3×500 mL). The organic layer was dried over sodium sulfate and evaporated to dryness using rotary evaporator to yield crude product. The crude material was purified by silica gel (60-120 mesh) column chromatography with 4% methanol-DCM. Pure fractions were combined and concentrated at 42° C. under reduced pressure to dryness to get tert-butyl (2-(piperazin-1-yl)ethyl)carbamate 18 (12.0 g, 48% yield) as light yellow oily liquid. ¹H NMR (CDCl₃, 400 MHz): δ 3.31-3.21 (m, 2H), 2.65 (m, 4H), 2.46 (m, 2H), 2.34 (m, 4H), 1.40 (s, 9H).

### Step 2: Synthesis of tert-butyl (2-(4-(piperazine-1-carbonyl)piperazin-1-yl)ethyl)carbamate

A 2000 ml three neck RB flask was equipped with a magnetic bar, 250 ml addition funnel and a N₂ inlet adapter. tert-butyl (2-(piperazin-1-yl)ethyl)carbamate 18 (12 g, 52.3 mmol, 1.0 eq) in DCM (800 ml) was added and stirred it for 10 minutes and cooled to 0° C. Then DIPEA (14.0 ml, 78.5 mmol, 1.5 eq) was added followed by triphosgene (4.7 g, 15.7 mmol, 0.30 eq) drop wise (dissolved in 100 ml of DCM) through addition funnel and the resulting reaction mixture was stirred at 0° C. for 1 h under N₂ atmosphere. The progress of the reaction monitored by TLC (10% methanol in dichloromethane). After complete consumption of tert-butyl (2-(piperazin-1-yl)ethyl)carbamate by TLC, N,N-diisopropylethylamine (14.0 ml, 78.5 mmol, 1.5 eq) was added and then piperazine (13.5 g, 157.0 mmol, 3.0 eq) in single portion and was allowed to stir at room temperature for 3 h. The reaction mixture was poured into ice water (500ml) and extracted with DCM (3 X 300 ml). The organic layer was separated and washed with water (3 X 1 L). the organic layer was dried over sodium sulfate and the reaction mixture was evaporated to dryness using rotary evaporator to yield crude product. Purification of the crude material using C18 (120 g) column chromatography using 40% Acetonitrile in water as eluent followed by combining the fractions containing pure product was lyophilized to get light brown solid which was triturated in diethylether: hexane (2:1). The precipitated solid was separated by decanting the solvent layer and dried evaporate at 40° C. under reduced pressure to dryness to yield tert-butyl (2-(4-(piperazine-1-carbonyl) piperazin-1-yl) ethyl) carbamate 19 (2.8 g, 16% yield) as light yellow solid. 1H NMR (D6-DMSO, 400 MHZ): δ 9.31 (brs, 1H), 6.73 (brs, 1H), 2.89-3.65 (m, 14H), 2.65-2.23 (m, 6H), 1.40 (s, 9H). LCMS (ESI, m/z): 342.1 (M+H) *.

Example 5. Preparation of a representative piperazine-pyridine/pyrimidine-alkyne linker

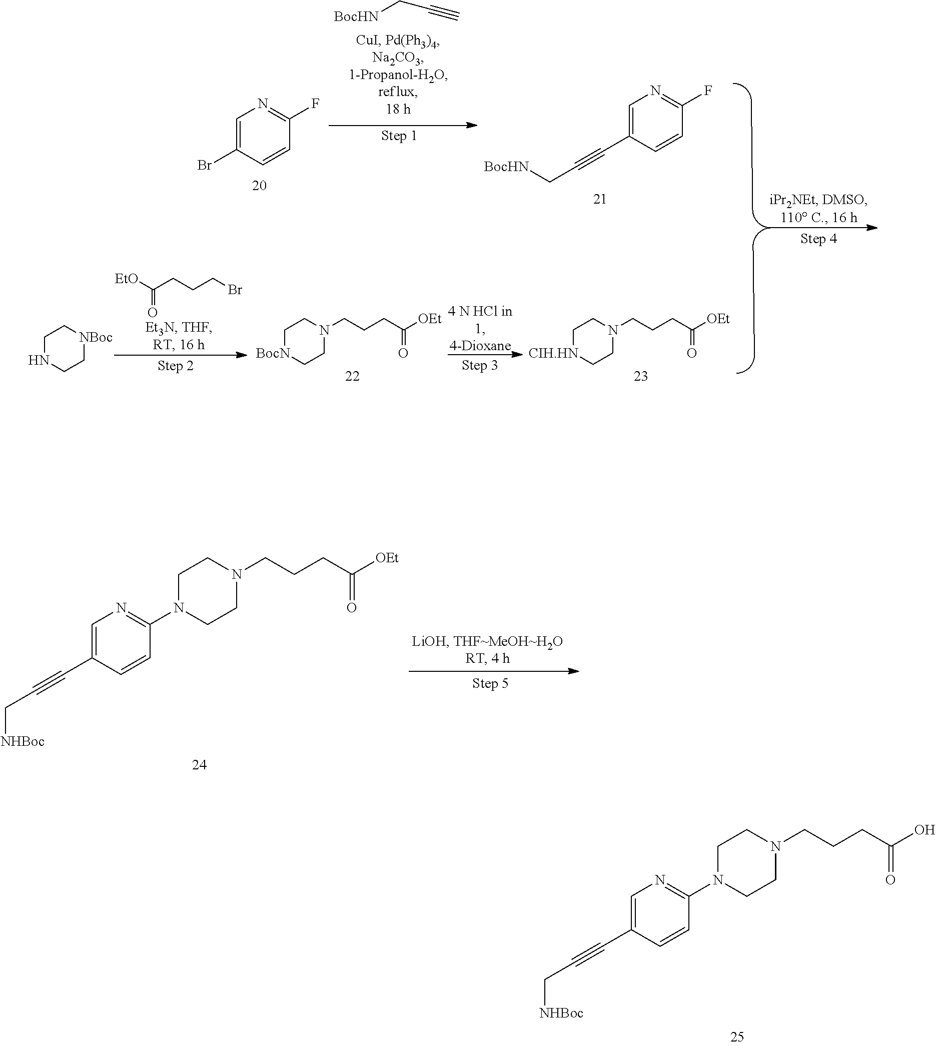

### Step 1: Synthesis of tert-butyl (3-(6-fluoropyridin-3-yl)prop-2-yn-1-yl)carbamate

A 2L RB flask was equipped with a magnetic bar, condenser and argon inlet adapter. 1-Propanol (500 mL) was charged and degassed using Argon for 60 min. N-Boc-propargylamine (44.2 g, 283.9 mmol, 1.0 eq) was added and degassed using Ar for 60 min. Next Copper (I) iodide (5.4 g, 28.4 mmol, 0.1 eq) was added and degassed using Ar for 30 min. Then tetrakis (triphenylphosphine) palladium (0) (16.4 g, 14.2 mmol, 0.05 eq) was added and degassed using Ar for 30 min. and stirred under Ar atmosphere.

Preparation of Aq. Sodium Carbonate Solution

A 250 mL 3-neck RB flask was equipped with a magnetic bar and an Ar inlet adapter. De-mineralized water (150 mL) was taken in the RB flask and degassed using Ar for 30 min. Then sodium carbonate (39.11 g, 369.1 mmol, 1.3 eq) was added to DM water, degassed with Ar for 30 min and stirred under Ar atmosphere to prepare aq. Solution of Sodium carbonate. The aq. solution of sodium carbonate was added to the reaction mixture in 2L RB flask and stirred under Ar atmosphere.

Preparation of Solution of 5-bromo-2-fluoropuridine in 1-propanol

A 250 mL 3-neck RB flask was equipped with a magnetic bar and an Ar inlet adapter. 1-Propanol (100 mL) was added to the RB flask and degassed using Ar for 30 min. 5-Bromo-2-fluoropuridine 20 (50 g, 283.9 mmol, 1.0 eq) was added, degassed using Ar for 30 min and stirred for 30 min. to prepare the solution.

Prepared solution was added to the reaction mass in 2 L RB flask and degassed using Ar for 30 min. Then the reaction mass was refluxed for 18 h under Ar atmosphere. The progress of reaction was monitored by TLC in 10% Ethyl acetate in hexane. The mass was cooled to RT and concentrated to dryness on rotary evaporator at 40-45° C. under reduced pressure. The residue was dissolved in 1 L of DCM and passed through a bed of anhydrous sodium sulphate. The bed was washed with DCM (500 mL). The filtrate was concentrated on rotary evaporator at 45° C. under reduced pressure to obtain the crude mass. The crude was purified by silica gel (100-200 mesh) column chromatography using Ethyl Acetate in Hexane as eluent. Pure fraction was collected in 3.5-5% Ethyl Acetate in Hexane. Pure fractions were combined and evaporated at 45° C. to obtain tert-butyl-{3-(6-fluoropyridin-3-yl) prop-2-yn-1-yl}carbamate 21 (36 g, 50% yield) as pale yellow liquid. ¹H NMR (CDCl₃, 400 MHz): δ 8.62 (s, 1H), 8.36 (m, 1H), 8.20 (brs, 1H), 7.23 (m, 1H), 3.97 (s, 2H), 1.40 (s, 9H).

### Step 2: Synthesis of tert-butyl 4-(4-ethoxy-4-oxobutyl)piperazine-1-carboxylate

A 1 L four neck RB flask was equipped with a magnetic bar, addition funnel and a N₂ inlet adapter. 1-Boc piperazine (38.0 g, 204.0 mmol, 1.0 eq) in 380 ml of THF was added at room temperature. Triethyl amine (34.5 ml, 244.8 mmol, 1.2 eq) was added dropwise using addition funnel over 20 minutes and then stirred for 30 minutes. Ethyl 4-bromobutyrate (41.8 g, 214.2 mmol, 1.05 eq) was added dropwise to the reaction mixture and stirred for 16 h at room temperature. The progress of reaction was monitored by TLC using 50% ethyl acetate in hexane. The reaction mass was diluted with ethyl acetate (1 L). To the above mass saturated NaHCO₃ solution was added, and layer was separated. The aq. layer was back extracted with Ethyl acetate. The combined organic layer was washed with brine solution (500 ml). The organic layer was dried over sodium sulphate and evaporated at 40-45° C. to obtained crude mass. The crude compound was purified by column chromatography using silica gel (100-200 mesh) using ethyl acetate in hexane as eluent. The pure fractions eluted with 35-50% ethyl acetate in hexane were collected and evaporated to afford tert-butyl 4-(4-ethoxy-4-oxobutyl)piperazine-1-carboxylate 22 (37 g, 61% yield) as pale brown oily liquid. ¹H NMR (CDCl₃, 400 MHZ): δ 4.07 (q, J=7.17 Hz, 2H), 3.21 (m, 4H), 2.48 (m, 8H), 1.89 (m, 2H), 1.40 (s, 9H), 1.18 (t, J=7.34, 3H).

### Step 3: Synthesis of ethyl 4-(piperazin-1-yl)butanoate hydrochloride

A 2 L four neck RB flask was equipped with a magnetic bar, 500 mL addition funnel, Nitrogen inlet adapter. Tert-butyl 4-(4-ethoxy-4-oxobutyl) piperazine-1-carboxylate 22 (37.5 g, 125.0 mmol) was added and 1,4-dioxane (375 ml) at room temperature. The mass was cooled to 10-15° C. 4M HCl in 1,4-dioxane (300 mL) was added dropwise using addition funnel over 45 minutes. The mass was warmed to RT and stirred for 5h. The progress of reaction was monitored by TLC in 10% methanol in dichloromethane. After completion of the reaction, the mass was transferred to 2 L Buchi flask and evaporated to dryness. To the obtained solid diethyl ether (2×300 mL) was added and evaporated to dryness. To the obtained solid was added 500 mL diethyl ether and filtered under nitrogen atmosphere. The solid was washed with (2×200 mL) diethyl ether. The off-white solid ethyl 4-(piperazin-1-yl) butanoate hydrochloride 23 (31 g, 99% yield) was collected under nitrogen atmosphere and dried under reduced pressure at 45° C. The crude compound was taken for the next step without further analysis.

### Step 4: Synthesis of ethyl 4-(4-(5-(3-((tert-butoxycarbonyl)amino) prop-1-yn-1-yl)pyridin-2-yl)piperazin-1-yl)butanoate

1 L three necked RB flask was equipped with a magnetic bar, dropping funnel and a N₂ inlet adapter. Ethyl 4-(piperazin-1-yl) butanoate hydrochloride 23 (25 g, 105.5 mmol, 1.0 eq) was added to DMSO (26 ml) at RT. DIPEA (90.0 ml, 527.5 mmol, 5.0 eq) was added to this and stirred for 20 minutes. Tert-butyl (3-(6-fluoropyridin-3-yl) prep-2-yn-1yl) carbamate 21 (26.4 g, 105.5 mmol, 1.0 eq) was added to reaction mixture. The resultant clear solution was heated to 110° C. and maintained overnight (16 h). Progress of the reaction was monitorred by TLC in 50% ethyl acetate: hexane. The reaction mass was poured to ice cold water (750 ml) and extracted with ethyl acetate (3×1000 ml). Combined organic layer was washed by water (1000 ml), saturated NaHCO₃ (500 ml) and brine (1000 ml), dried over sodium sulphate and evaporated under reduced pressure to obtained crude product. Purification of the crude material using silica gel (100-200 mesh) column chromatography using 50% ethyl acetate in hexane as eluent afforded ethyl 4-(4-(5-(3-((tert-butoxycarbonyl)amino) prop-1-yn-1-yl)pyridin-2-yl)piperazin-1-yl)butanoate 24 (28 g, 62% yield). ¹H NMR (CDCl₃, 400 MHz): δ 8.62 (s, 1H), 8.36 (m, 1H), 8.20 (brs, 1H), 7.23 (m, 1H), 4.07 (q, J=7.17 Hz, 2H), 3.97 (s, 2H), 3.21 (m, 4H), 2.48 (m, 8H), 1.89 (m, 2H), 1.40 (s, 9H), 1.18 (t, J=7.34, 3H).

### Step 5: Synthesis of 4-(4-(5-(3-((tert-butoxycarbonyl)amino)prop-1-yn-1-yl)pyridin-2-yl)piperazin-1-yl)butanoic acid

A 500 ml three necked RB flask was equipped with a magnetic bar, addition funnel and a N₂ inlet adapter. 4-(4-(5-(3-((tert-butoxycarbonyl) amino)prop-1-yn-1-yl)pyridine-2-yl)piperazin-1-yl)butanoate 24 (18.0 g, 41.8 mmol, 1.0 eq), THF (144 ml) and Methanol (36 ml) were added into the reaction flask at room temperature. To the above solution added aqueous solution of lithium hydroxide monohydrate (3.5 g LiOH in 72 mL water, 83.6 mmol, 2.0 eq). The reaction mixture was stirred at room temperature for 4 h. Progress of reaction monitored by TLC using 20% methanol in dichloromethane. The reaction mass was diluted with ethyl acetate (250 ml) and layer separated. The Aq. layer was washed with ethyl acetate (250 mL). Organic layer was discarded. The pH of aq. layer was adjusted to 5.5 to 5.7 using Amberlyst acidic resin (Amberlyst IR 120 Hydrogen form). During acidification solid precipitated out. The solid was filtered and washed with acetone (100 mL). Then the solid was purified by reverse phase column chromatography using acetonitrile in water as eluent. Pure fractions were concentrated in lyophilizer to afford 4-(4-(5-(3-((tert-butoxycarbonyl)amino)prop-1-yn-1-yl)pyridin-2-yl)piperazin-1-yl)butanoic acid 25 (10.9 g, 65%) as off-white solid. ¹H NMR (D₆-DMSO, 400 MHz): δ 8.64 (s, 1H), 8.37 (m, 1H), 8.23 (brs, 1H), 7.21 (m, 1H), 3.97 (s, 2H), 3.21 (m, 4H), 2.46 (m, 8H), 1.88 (m, 2H), 1.41 (s, 9H).

### Example 6. Preparation of a Representative Pyridine-PEG-Piperazine Linker

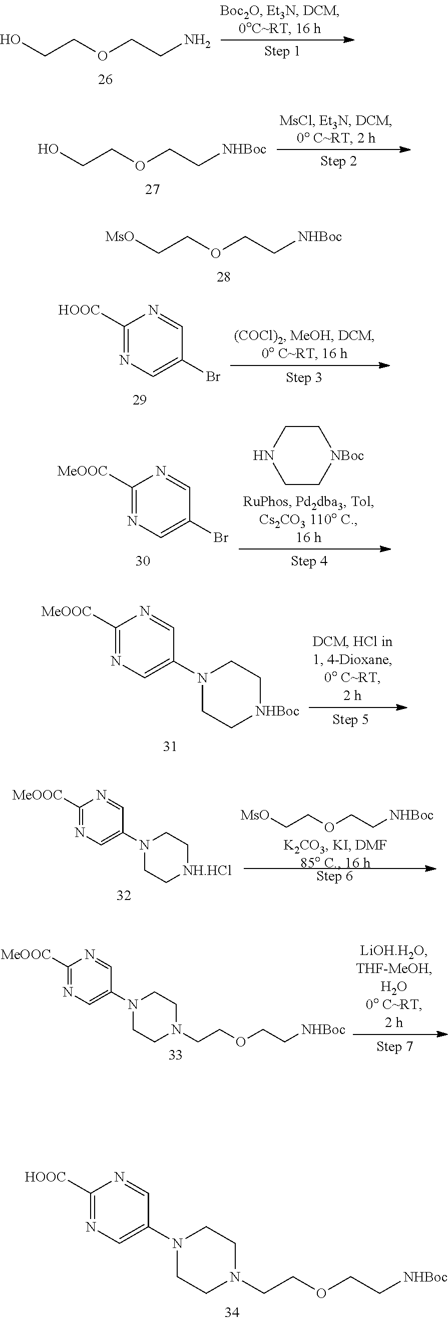

### Step 1: Synthesis of 1,1-dimethylethyl N-[2-(2-hydroxyethoxy)ethyl]carbamate

2-(2-Aminoethoxy) ethanol 26 (20.0 g, 190.22 mmol, 1.0 eq) was dissolved in 100 mL of methylene chloride and triethylamine (53.0 ml, 380.44 mol, 2.0 eq) was added to it. The solution was cooled in an ice-bath and di-tert-butyl dicarbonate (62.27 g, 285.33 mol, 1.5 eq) in 60 mL of methylene chloride was added through an additional funnel. The reaction was slowly warmed to room temperature and stirred at room temperature for overnight. After completion (checked by TLC, 80% EtOAc in hexane, R_{f}=0.30), the reaction was washed with water and the organic phase was washed with diluted HCl solution (0.5 N), water (100 ml), brine and dried over sodium sulfate. Solvent was evaporated and the crude product was purified by column chromatography to afford 1,1-dimethylethyl N-[2-(2-hydroxyethoxy)ethyl]carbamate 27 (25 g, 64% yield) as oily liquid. 1H (400 MHz, CDCl₃) δ 5.28 (brs, 1H), 3.74 (m, 2H), 3.59-3.54 (m, 4H), 3.32-3.28 (m, 2H), 3.08 (brs, 1H), 1.44 (s, 9H).

### Step 2: Synthesis of 2-[2-[(tert-butoxycarbonyl)amino]ethoxy]ethyl methanesulfonate

To a stirred solution of 1,1-Dimethylethyl N-[2-(2-hydroxyethoxy)ethyl]carbamate 27 (10.0 g, 48.7 mmol, 1.0 eq) in DCM (100 ml) was added triethylamine (10.2 ml, 73.0 mmol, 1.5 eq) and methanesulfonyl chloride (5.6 ml, 73.0 mmol, 1.5 eq) at 0° C. under inert atmosphere. The reaction mixture was warmed to ambient temperature and reaction progress was monitored by TLC (30% EtOAc-Hexane). After completion, the reaction mixture was diluted with DCM (200 ml), washed with water (100 ml×2) and brine and dried over sodium sulfate. The organic layer was concentrated under reduced pressure to yield 2-[2-[(tert-butoxycarbonyl) amino] ethoxy] ethyl methanesulfonate 28 as oily liquid (13.5 g, 98% yield) and used for the next step without further purification. ¹H NMR (CDCl₃, 400 MHz): δ 4.83 (brs, 1H), 4.30 (m, 2H), 3.66 (m, 2H), 3.49 (m, 2H), 3.25 (m, 2H), 2.99 (s, 3H), 1.37 (s, 9H).

### Step 3: Synthesis of 5-bromo-2-pyrimidinecarboxylic acid methyl ester

To a stirred suspension of 5-bromopyrimidine-2-carboxylic acid 29 (25.0 g, 123.2 mmol, 1.0 eq) in DCM (250 ml) was added oxalyl chloride (15.6 ml, 184.73 mmol, 1.5 eq) at 0-5° C. dropwise for 20 minutes. The reaction mixture was stirred at 0-5° C. for 10 min followed by addition of DMF (0.2 mL). The reaction mixture was gradually warmed to ambient temperature and stirred for 5 h. The reaction mixture was again cooled to 0-5° C. and methanol (200 ml) was added over a period of 20 min. The reaction mixture was warmed to room temperature and stirred for overnight. Progress of the reaction was monitored by TLC (5% MeOH in DCM, visualization by UV) and concentrated under reduced pressure after complete conversion. The crude reaction mixture was diluted with DCM (1 L) and washed with saturated sodium bicarbonate solution (500 mL), water (500 ml), brine and the organic layer was dried over Na₂SO₄, filtered and concentrated to afford 5-bromo-2-pyrimidinecarboxylic acid methyl ester 30 (17.0 g, 63% yield) as white solid. ¹H NMR (CDCl₃, 400 MHz): δ 8.91 (s, 2H), 4.00 (s, 3H).

### Step 4: Synthesis of methyl 5-[4-[(1,1-dimethylethoxy)carbonyl]-1-piperazinyl]-2-pyrimidinecarboxylate

Toluene (1350 ml) was added to a 2 L 4-neck round-bottom flask equipped with Argon inlet, thermo-pocket, condenser and purged with gentle blow Ar for 1 h. 5-bromo-2-pyrimidinecarboxylic acid methyl ester 30 (27.0 g, 124.42 mmol, 1.0 eq), 1-Boc-piperazine (29.0 g, 155.53 mmol, 1.25 eq) and Cs₂CO₃ (121.6 g, 373.26 mmol, 3.0 eq) were added and Ar was purged for 1 h. RuPhos (5.80 g, 12.44 mmol, 0.1 eq) and tris(dibenzylideneacetone)dipalladium(0) (2.85 g, 3.11 mmol, 0.025 eq) were added at RT and Ar was purged for 1 h. The reaction mixture was stirred at 115° C. for 16 h. After completion of reaction (monitored by TLC, 5% MeOH in DCM, visualized by UV) the reaction mixture was filtered through a pad of celite and washed with 10% MeOH in EtOAc (1 L). The filtrate was concentrated under reduced pressure and the residue was dissolved again in ethyl acetate (1 L). The organic layer was washed with saturated sodium bicarbonate solution (500 mL), water (500 ml), and brine. The organic layer was dried over Na₂SO₄, filtered, and then concentrated under reduced pressure. The crude compound was purified by silica gel chromatography to afford Methyl 5-[4-[(1,1-dimethylethoxy)carbonyl]-1-piperazinyl]-2-pyrimidinecarboxylate 31 (17.0 g, 42% yield) as white solid. ¹H NMR (D₆-DMSO, 400 MHz): δ 8.46 (s, 2H), 3.76 (m, 2H), 3.54 (m, 2H), 3.23 (m, 4H), 2.65 (s, 3H), 1.86 (s, 9H).

### Step 5: Synthesis of methyl 5-(piperazin-1-yl)pyrimidine-2-carboxylate hydrochloride

To a stirred suspension of methyl 5-[4-[(1,1-dimethylethoxy)carbonyl]-1-piperazinyl]-2-pyrimidinecarboxylate 31 (15 g, 46.53 mmol, 1.0 eq) in dichloromethane (300 ml) was added 4 N HCl in 1,4-dioxane (150 ml) at 0° C. under inert atmosphere. The reaction mass was allowed to warm to room temperature and stirred at rt for 2 h. The progress of the reaction was monitored by TLC (10% MeOH in DCM, UV visualization) and the reaction mass was concentrated under reduced pressure after reaction completion. The residue was triturated with diethyl ether (200 ml×2) to afford greenish colored solid, methyl 5-(piperazin-1-yl) pyrimidine-2-carboxylate hydrochloride 32 (12 g, 99% crude yield). The compound was taken to next step without further analysis.

### Step 6: Synthesis of methyl 5-(4-(2-(2-((tert-butoxycarbonyl)amino)ethoxy)ethyl)piperazin-1-yl)pyrimidine-2-carboxylate

To a stirred suspension of methyl 5-(piperazin-1-yl)pyrimidine-2-carboxylate hydrochloride 32 (12.0 g, 46.4 mmol, 1.0 eq) in DMF (200 ml) were added 2-[2-[(tert-butoxycarbonyl)amino]ethoxy]ethyl methanesulfonate 28 (19.7 g, 69.6 mmol, 1.5 eq), K₂CO₃ (16.0 g, 116.2 mmol, 2.5 eq) & KI (0.8 g, 4.6 mmol, 0.1 eq) at room temperature under inert atmosphere. The reaction mixture was heated at 85° C. for 16 h. The progress of the reaction was monitored by TLC (10% MeOH in DCM, UV visualisation) and after completion, the reaction mass was concentrated under reduced pressure. Water (200 ml) was added to the residue and the mixture was extracted with ethyl acetate (200 ml×2). The organic layers were combined, dried over anhydrous sodium sulfate, filtered and then concentrated under reduced pressure to afford methyl 5-(piperazin-1-yl)pyrimidine-2-carboxylate hydrochloride 33 (12 g, 99% crude yield) as green colored solid. The compound was taken to next step without further analysis.

### Step 7: Synthesis of methyl 5-(4-(2-(2-((tert-butoxycarbonyl)amino)ethoxy)ethyl)piperazin-1-yl)pyrimidine-2-carboxylic acid

A 500 ml three necked RB flask was equipped with a magnetic bar, addition funnel and a N₂ inlet adapter. Methyl 5-(piperazin-1-yl)pyrimidine-2-carboxylate hydrochloride 33 (4.0 g, 9.8 mmol, 1.0 eq), THF (14.4 ml) and methanol (3.6 ml) were added into the reaction flask at room temperature. To the above solution added LiOH solution (820 mg LiOH in 7.2 mL water, 19.6 mmol, 2.0 eq) dropwise. The reaction mixture was stirred at room temperature for 4 h. Progress of reaction monitored by TLC using 20% methanol in dichloromethane. The reaction mass was diluted with ethyl acetate (250 ml) and two layers were separated. The aqueous layer was washed with ethyl acetate (250 mL). The pH of aqueous layer was adjusted to 5.5 to 5.7 using Amberlyst acidic resin (Amberlyst IR 120Hydrogen form). During acidification, precipitated solid was filtered and washed well with acetone (100 mL). The solid was purified by reverse phase column chromatography to afford methyl 5-(4-(2-(2-((tert-butoxycarbonyl)amino)ethoxy)ethyl)piperazin-1-yl)pyrimidine-2-carboxylic acid 34 (3.3 g, 85%) as off-white solid. ¹H NMR (D₆-DMSO, 400 MHz): δ 11.96 (brs, 1H), 8.76 (s, 2H), 6.76 (brs, 1H), 3.61-3.40 (m, 8H), 3.09 (m, 6H), 2.76 (m, 2H), 1.86 (s, 9H).

### Example 7. Preparation of a Representative Pyrimidine-Piperazine-PEG Linker

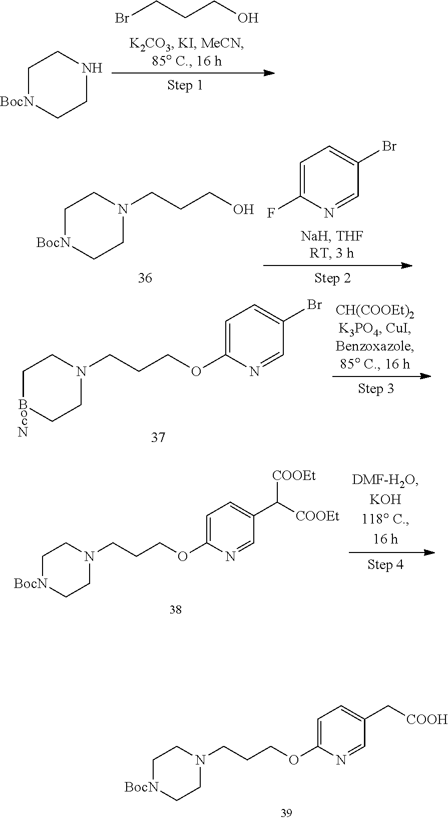

### Step 1: Synthesis of tert-butyl 4-(3-hydroxypropyl)piperazine-1-carboxylate

1-Boc-piperazine (25.0 g, 134.2 mmol, 1.0 eq), K₂CO₃ (46.3 g, 335.5 mmol, 2.5 eq), KI (2.2 g, 13.4 mmol, 0.1 eq) were taken in a 500 ml three neck RB flask in acetonitrile (250 ml) under inert atmosphere and 3-Bromo-1-propanol (20.5 g, 147.6 mmol, 1.1 eq) in 100 ml acetonitrile was added into it. The reaction mixture was reflux for overnight and completion of reaction was checked by TLC (5% MeOH-DCM, Visualization: PMA, R_{f} 0.25). The reaction mixture was concentrated and then extracted with ethyl acetate (500 ml). The organic layer was washed with water and brine. The ethyl acetate layer was dried over Na₂SO₄, filtered and then concentered under reduced pressure. The crude material was purified by column chromatography (silica gel, 2% methanol in DCM) to afford tert-butyl 4-(3-hydroxypropyl) piperazine-1-carboxylate 36 (25 g, 76% yield) as off-white solid. ¹H NMR (CDCl₃, 400 MHZ): δ 3.50 (m, 2H), 3.19 (m, 4H), 2.43 (m, 6H), 1.59 (m, 2H), 1.81 (s, 9H).

### Step 2: Synthesis of tert-butyl 4-(3-((5-bromopyridin-2-yl)oxy)propyl)piperazine-1-carboxylate

Tert-butyl 4-(3-hydroxypropyl)piperazine-1-carboxylate 36 (20.0 g, 113.9 mmol, 1.0 eq) was taken in THF (300 ml) under inert atmosphere and NaH (60% dispersion in mineral oil, 5.4 g, 134.4 mmol, 1.18 eq) was added in portions. The reaction mixture was stirred for 0.5 h and 5-Bromo-2-fluoropyridine (23.0 g, 131.0 mmol, 1.15 eq) in THF (50 ml) was added dropwise into the reaction mixture at 0° C. The reaction mixture was warmed to room temperature completion of reaction was checked by TLC (40% ethyl acetate in hexane, R_{f} 0.5). The reaction mixture was quenched with cold saturated NH₄Cl solution. Solvents and volatiles were removed under reduced pressure. The residue was extracted with ethyl acetate (500 ml×1). The organic layer was washed with water, brine and then dried over Na₂SO₄. The solvent was concentrated under reduced pressure. The crude material was purified by column chromatography (silica gel, ethyl acetate-hexane) to afford tert-butyl 4-(3-((5-bromopyridin-2-yl)oxy)propyl)piperazine-1-carboxylate 37 (25 g, 76% yield) as off-white solid. ¹H NMR (CDCl₃, 400 MHz): δ 8.21 (s, 1H), 7.53 (d, 1H), 6.57 (d, 1H), 4.30 (m, 2H), 3.43 (m, 4H), 2.51 (m, 2H), 2.39 (m, 4H), 1.89 (m, 2H), 1.43 (s, 9H).

### Step 3: Synthesis of diethyl 2-(6-(3-(4-(tert-butoxycarbonyl)piperazin-1-yl)propoxy)pyridin-3-yl)malonate

tert-Butyl 4-(3-((5-bromopyridin-2-yl)oxy)propyl)piperazine-1-carboxylate 37 (33.0 g, 82.4 mmol, 1.0 eq) was taken in DMSO (330 ml) under inert atmosphere and K₃PO₄ (52.5 g, 247.2 mmol, 3.0 eq), CuI (1.56 g, 8.2 mmol, 0.1 eq), benzoxazole (1.95 g, 16.4 mmol, 0.2 eq) were added and the solution was purged with Ar for 0.5 h. Diethylmalonate (18.8 mL, 123.6 mmol, 1.5 eq) was added and the reaction mixture was stirred at 85° C. for 18 hours. Completion of reaction was checked by TLC (70% EtOAc-Hexane, R_{f} 0.4). The reaction mixture was poured into ice-cold water and extracted with EtOAc (500 ml). The organic layer was washed with water, brine and dried over Na₂SO₄. The solvent was concentrated and purified by column chromatography (silica gel, EtOAc-Hexane) to afford diethyl 2-(6-(3-(4-(tert-butoxycarbonyl)piperazin-1-yl)propoxy)pyridin-3-yl)malonate 38 (24 g, 61% yield) as pale-yellow liquid. ¹H NMR (CDCl₃, 400 MHz): δ 8.21 (s, 1H), 7.53 (d, 1H), 6.57 (d, 1H), 4.57 (s, 1H), 4.30 (m, 2H), 4.23 (q, 4H), 3.43 (m, 4H), 2.51 (m, 2H), 2.39 (m, 4H), 1.89 (m, 2H), 1.43 (s, 9H), 1.30 (t, 6H).

### Step 4: Synthesis of 2-(6-(3-(4-(tert-butoxycarbonyl)piperazin-1-yl)propoxy)pyridin-3-yl)acetic acid

Diethyl 2-(6-(3-(4-(tert-butoxycarbonyl)piperazin-1-yl)propoxy)pyridin-3-yl)malonate 38 (24.0 g, 50.1 mmol, 1.0 eq) was taken in DMF (190 ml) and KOH (28.1 g, 501.0 mmol, 10.0 eq, dissolved in 48 ml water) was added into it. The reaction mixture was stirred for 18 h at 115° C. Completion of reaction was checked by TLC (5% MeOH-DCM, R_{f} 0.2). The reaction mixture was poured into ice-cold water and extracted with EtOAc (500 ml×2). The organic layer was washed with water, brine and dried over Na₂SO₄. The solvent was concentrated and the residue was purified by column chromatography (silica gel, EtOAc-Hexane) to afford 2-(6-(3-(4-(tert-butoxycarbonyl) piperazin-1-yl)propoxy)pyridin-3-yl)acetic acid 39 (15 g, 78% yield) as off-white solid. ¹H NMR (D₆-DMSO, 400 MHz): δ 8.21 (s, 1H), 7.53 (d, 1H), 6.57 (d, 1H), 4.30 (t, 2H), 3.67-3.43 (m, 6H), 2.51 (m, 2H), 2.39 (m, 4H), 1.89 (m, 2H), 1.43 (s, 9H).

### Example 8. First Representative Example of Linking Linker B to E3 Ligase Ligand C

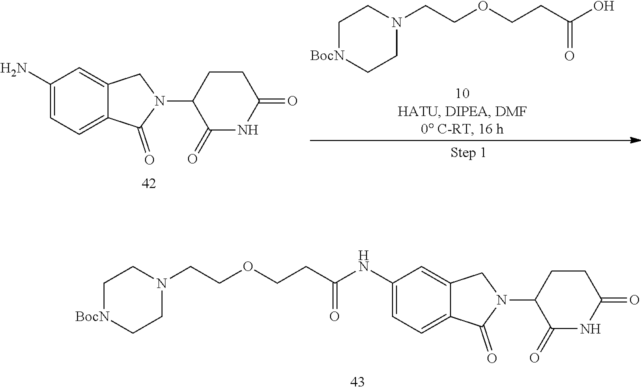 [SMILES: Nc1ccc2c(c1)CN(C1CCC(=O)NC1=O)C2=O ; CC(C)(C)OC(=O)N1CCN(CCOCCC(=O)O)CC1 ; CC(C)(C)OC(=O)N1CCN(CCOCCC(=O)Nc2ccc3c(c2)CN(C2CCC(=O)NC2=O)C3=O)CC1]

### Step 1: Synthesis of 4-(2-(2-(ethoxycarbonyl)ethoxy)ethyl)piperazine-3-(1-oxoisoindolin-2-yl)piperidine-2,6-dione-5-carboxamide

An oven dried 250 mL three neck RB flask was equipped with a magnetic bar, thermometer with a N₂ inlet adapter. The system with was flushed with N₂ for 10 minutes and added 4-(2-(2-(ethoxycarbonyl)ethoxy)ethyl)piperazine-1-carboxylic acid 10 (4.19 g, 13.8 mmol, 1.2 eq) and DMF (75 ml) with stirring. To this solution then HATU (8.80 g, 23.0 mmol, 2.0 eq.) was added and was stirred at room temperature for 10-15 minutes. The reaction mixture cooled to 0° C. using ice bath. C5-Lenalidomide 42 (3.0 g, 11.5 mmol, 1.0 eq) and N-ethyl-diisopropyl amine (8.0 ml, 46.0 mmol, 4.0 eq) were added and allowed to stir the reaction mixture room temperature for 16 hours. The completion of reaction was checked by TLC (5% MeOH in, R_{f}=0.3). The reaction mixture was poured into ice cold water and extracted with ethyl acetate (600 ml). The organic layer was washed with sat. NaHCO₃ solution (250 ml) and brine solution (300 ml), dried over Na₂SO₄ and evaporated on rotary evaporator to yield crude product as a gummy liquid. Purification of crude in silica gel (10-200 mesh) column using 10% MeOH in dichloromethane afforded light brown solid 4-(2-(2-(ethoxycarbonyl)ethoxy)ethyl)piperazine-3-(1-oxoisoindolin-2-yl)piperidine-2,6-dione-5-carboxamide. Repurification of this column pure material through reverse phase column chromatography using C18 column having an eluent Water: Acetonitrile afforded 1.2 g of pure 4-(2-(2-(ethoxycarbonyl)ethoxy)ethyl)piperazine-3-(1-oxoisoindolin-2-yl)piperidine-2,6-dione-5-carboxamide (29% yield). ¹H NMR (D₆-DMSO, 400 MHz): δ 11.00 (s, 1H), 10.43 (s, 1H), 9.01 (brs, 1H), 8.05 (s, 1H), 7.67 (s, 1H), 5.01 (m, 1H), 4.41 (d, 1H), 4.29 (m, 1H), 3.65 (t, 4H), 3.43 (t, 4H), 2.72 (t, 2H), 2.52 (t, 4H), 2.45 (t, 2H), 1.43 (s, 9H).

### Example 9. Second Representative Example of Linking Linker B to E3 Ligase Ligand C

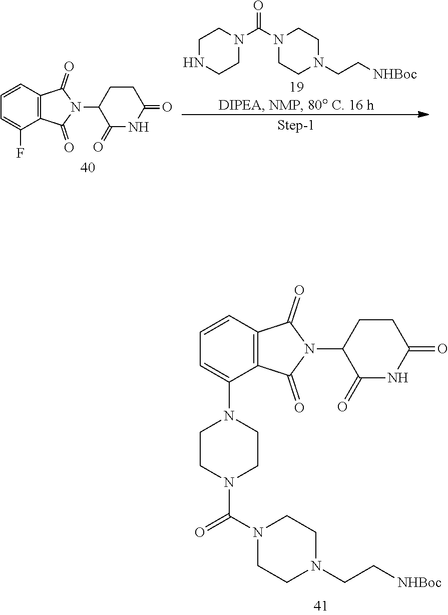 [SMILES: O=C1CCC(N2C(=O)c3cccc(F)c3C2=O)C(=O)N1 ; CC(C)(C)OC(=O)NCCN1CCN(C(=O)N2CCNCC2)CC1 ; CC(C)(C)OC(=O)NCCN1CCN(C(=O)N2CCN(c3cccc4c3C(=O)N(C3CCC(=O)NC3=O)C4=O)CC2)CC1]

### Step 1: tert-butyl (2-(4-(4-(2-(2,6-dioxopiperidin-3-yl)-1,3-dioxoisoindolin-4-yl)piperazine-1-carbonyl)piperazin-1-yl)ethyl)carbamate

A 250 mL three necked RB flask was equipped with a magnetic bar, refluxing condenser and a N₂ inlet adapter. 2-(2,6-dioxopiperidin-3-yl)-4-fluoroisoindoline-1,3-dione 40 (3.0 g, 10.0 mmol, 1.0 eq) in 50 ml of NMP and tert-butyl (2-(4-(piperazine-1-carbonyl)piperazin-1-yl)ethyl)carbamate 19 (4.45 g, 13.0 mmol, 1.3 eq) followed by DIPEA (5.6 ml, 32.0 mmol, 3.2 eq) were added into the reaction flask with stirring. The reaction mixture was stirred at room temperature for 20 minutes followed by 80° C. for 16 hours. The reaction progress was monitored by TLC in 5% MeOH: DCM. The reaction mixture was cooled to room temperature and poured into ice water (250 ml) and extract with ethyl acetate (3×200 ml). The organic layer was dried over sodium sulfate and evaporated to dryness using rotary evaporator to yield crude product. Purification of the crude material using silica gel (230-400 mesh size) column chromatography using 60% Acetone in Hexane as eluent. Pure fractions were evaporated to yield yellow solid tert-butyl (2-(4-(4-(2-(2,6-dioxopiperidin-3-yl)-1,3-dioxoisoindolin-4-yl)piperazine-1-carbonyl)piperazin-1-yl)ethyl)carbamate 41 (1.2 g, 18% yield). 1H NMR (CDCl₃, 400 MHz): δ 11.31 (brs, 1H), 11.01 (s, 1H), 8.53 (brs, 1H), 7.87 (m, 1H), 7.37 (m, 2H), 5.05 (m, 1H), 3.76-3.57 (m, 4H), 2.89-3.65 (m, 14H), 2.65-2.23 (m, 6H), 1.40 (s, 9H).

The examples herein are for illustrative purposes and are not meant to limit the scope of the invention as set forth in the claims.

1. A linker of Formula I 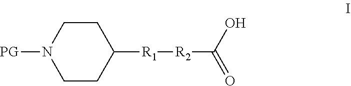
wherein
R₁ is optional, and if present, is selected from piperidine and piperazine,
R₂ is selected from the group consisting of C₁-C₈ alkane and C₂-C₈ alkyne, and PG is an amine protecting group.

2. The linker of claim 1 wherein R₂ is selected from the group consisting of ethane and ethyne.

3. The linker of claim 1 wherein PG is tert-butoxycarbonyl (Boc).

4. The linker of claim 1 wherein the linker is selected from

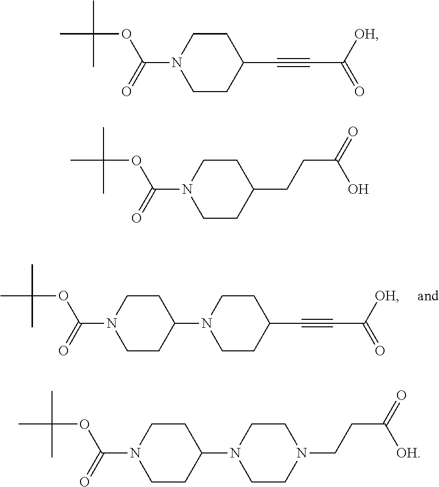

5. A linker of Formula II: 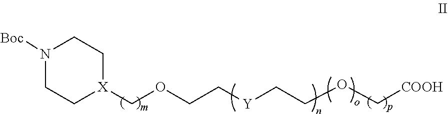
wherein
X is selected from C and N,
m is 1 to 6,
Y is selected from —O— and —CH₂—,
n is from 0 to 4,
o is 0 or 1, and
p is 0 to 9.

6. The linker of claim 5, wherein the linker is of Formula IIa: 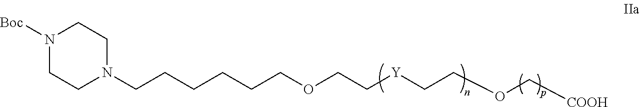
wherein
Y is selected from —CH₂— and —O—;
n is 0 to 5, and
p is 1 to 5.

7. The linker of claim 6, wherein n is 0, 1, or 4.

8. The linker of claim 7 wherein p is 1 or 5.

9. The linker of claim 5, wherein the linker is that of Formula IIb: 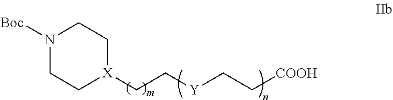
wherein
X is selected from C and N,
Y is selected from —CH₂— and —O—;
m is 0 or 1, and
n is 0, 1, 2, or 3.

10. A linker of Formula IIc: 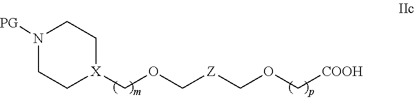
wherein
X is selected from the group consisting of C and N;
Z is selected from the group consisting of —CH₂—, —CF₂—, —C(O)—, and —C(S)—;
m is 2 to 5;
p is 1 or 2; and
PG is an amine protecting group.

11. The linker of claim 10 wherein m is 2, 3 or 5.

12. The linker of claim 11, wherein PG is Boc.

13. A linker of Formula III: 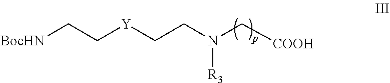
wherein
Y is selected from —O— and —CH₂—;
R₃ is selected from the group consisting of C₁-C₆ alkyl, C₃-C₆ cycloalkyl, and cycloalkyl-substituted alkyl, and
p is 1 or 2.

14. The linker of claim 13 wherein R₃ is selected from the group consisting of methyl, ethyl, isopropyl, tert-butyl, n-butyl, cyclopropyl, cyclobutyl, cyclopentyl, cyclohexyl, and methylenecyclobutyl

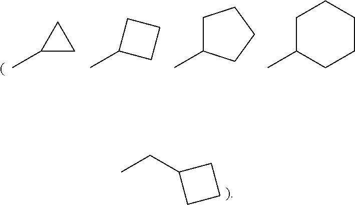

15. A linker of Formula IV: 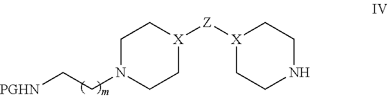
wherein
PG is an amine protecting group;
m is 1-4;
each X is independently selected from C and N; and
Z is selected from the group consisting of —C(O)—, —C(S)—, —CH₂—, and —CF₂—.

16. The linker of claim 15 wherein PG is Boc.

17. A linker of Formula V: 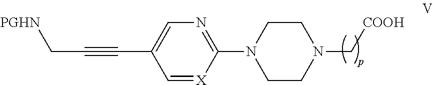
wherein
PG is an amine protecting group,
X is selected from N and C, and
p is 1-3.

18. The linker of claim 17 wherein PG is Boc.

19. A linker of Formula VI: 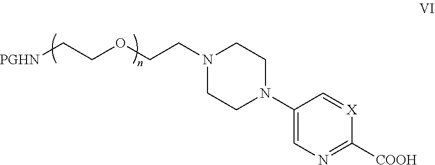
wherein
PG is an amine protecting group;
each X is independently selected from C and N; and
n is 0-2.

20. The linker of claim 19 wherein PG is Boc.

21. A linker of Formula VII: 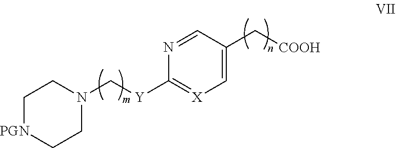
wherein
PG is an amine protecting group;
X is selected from C and N;
Y is selected from N, O and C;
m is 1-3; and
n is 1-3.

22. The linker of claim 21, wherein PG is Boc.

23. A compound comprising a linker of claim 21 and a ligand that triggers a degradation pathway.

24. The compound of claim 23 wherein the ligand that triggers a degradation pathway is an E3 ubiquitin ligase enzyme.

25. The compound of claim 24, wherein the ligand for an E3 ubiquitin ligase enzyme is a derivative of an immunomodulatory drug (IMiD).

26. The compound of claim 25 wherein the derivative of an IMiD is selected from the group consisting of pomalidomide derivatives, thalidomide derivatives, lenalodomide derivatives, and VH032 derivatives.

27. The linker of claim 1 wherein R₁ is absent, R₂ is C₂-C₈ alkyne, and PG is Boc.

28. The linker of claim 15 wherein PC is Boc, X is N, and PG is Boc.

29. The linker of claim 18 wherein X is C.

30. The linker of claim 21, wherein the linker is 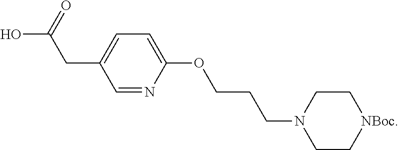 [SMILES: CC(C)(C)OC(=O)N1CCN(CCCOc2ccc(CC(=O)O)cn2)CC1]
